(19) United States
(12) Patent Application Publication
(10) Pub. No.: US 2017/0367591 A1
(43) Pub. Date: Dec. 28, 2017

(54) WEARABLE CARDIOVERTER DEFIBRILLATOR (WCD) APPARATUS AND METHOD FOR IMPROVED COMFORT AND LONGER WEAR

(71) Applicant: KONINKLIJKE PHILIPS N.V., EINDHOVEN (NL)

(72) Inventor: DAWN BLILIE JORGENSON, MERCER ISLAND, WA (US)

(21) Appl. No.: 15/535,581

(22) PCT Filed: Dec. 18, 2015
(86) PCT No.: PCT/IB2015/059764

§ 371 Date: Jun. 13, 2017

Related U.S. Application Data
(60) Provisional application No. 62/093,495, filed on Dec. 18, 2014.

(51) Int. Cl. A61B 5/0205; A61B 5/00; A61B 5/0408; A61B 5/046; A61N 1/39; A61N 1/04; A61B 5/0464; A61B 5/11; A61B 5/024; A61B 5/08; A61N 1/372

(52) U.S. Cl. CPC A61B 5/0205; A61N 1/046; A61B 5/6805; A61B 5/04085; A61B 5/046; A61B 5/0464; A61N 1/39; A61B 5/02438; A61B 5/02416; A61B 5/0816; A61B 5/0002; A61B 5/7405; A61N 1/0484; A61N 1/37258; A61N 1/3993; A61B 5/1107

(57) ABSTRACT

A wearable cardioverter defibrillator (WCD) (10) and method (60) comprise a set of electrodes (12) for placement on a subject (14), a mechanism for electrically engaging (16) the set of electrodes to the subject's skin, and at least one non-invasive physiologic sensor (18, 20) configured for placement on the subject. A controller (24) monitors an output of the non-invasive physiologic sensor (18, 20) for detecting a change in a health parameter of the subject being indicative of one or more of a change in subject condition that may be a precursor to potential cardiac arrhythmia or a simultaneously occurring cardiac arrhythmia. Responsive to detecting the change, the controller (24) activates an alarm (26) for requesting a response from the subject (14) within a predetermined time. Responsive to receiving the subject's response within the predetermined time, the controller (24) inhibits the mechanism (16) from electrically engaging the set of electrodes (12) to the subject's skin. Responsive to not receiving the subject's response, the controller (24) initiates the mechanism (16) for electrically engaging the set of electrodes (12) to the subject's skin.

The present embodiments relate generally to wearable cardioverter defibrillator (WCD) apparatus and more particularly, to a WCD apparatus featuring patient health status detection for improved comfort and longer wear, further for potentially easier and more reliable detection of a change in patient condition that may signal or differentiate a need for further analysis (e.g., ECG) and a method thereof

At least one known wearable cardioverter defibrillator (WCD) currently on the market uses two sets of electrodes. One set of electrodes comprise dry sensing electrodes for ECG assessment and shock determination. The other set of electrodes is for application of a therapeutic shock. If the sensing electrodes identify a shockable rhythm, the therapeutic electrodes are deployed thru exploding gel. However, with the known WCD, it is well known that the average patient with the WCD only wears it about 5-6 hours a day due to discomfort associated with wearing the known WCD. In particular, patients wearing the known WCD suffer with messy or uncomfortable electrodes on their skin on a continuous basis, which has been a significant compliance problem with existing wearable defibrillators.

Accordingly, an improved method and apparatus for overcoming the problems in the art is desired.

In one embodiment of the present disclosure, a method is disclosed for an improvement to alerting of the WCD system to a potential arrhythmia and for providing more comfortable wear for the patient. The embodiments of the present disclosure also advantageously provide an advancement that improves a wearability of a WCD, which promotes increased wear time of the WCD, and thereby increasing a safety to the patient.

The embodiments of the present disclosure further relate to wearable defibrillators which activate electrode contact in response to non-electrode sensors. The sensors include accelerometers and blood oxygen detectors (e.g. photoplethysmographic detectors). In one embodiment, an advanced Wearable Cardioverter Defibrillator with increased wear time uses non-electrode sensors, in particular, blood oxygen detectors (e.g. photo-plethysmographic detectors) and accelerometer sensors to activate a therapy electrode.

According to one embodiment, a wearable cardioverter defibrillator (WCD) comprises a set of electrodes configured for placement on a subject, the set of electrodes at least operable to sense an ECG signal from the subject. The WCD further comprises a means for electrically engaging the set of electrodes to the subject's skin. At least one non-invasive physiologic sensor is configured for placement on the subject, wherein the at least one non-invasive physiologic sensor comprises one or more of a photoplethysmographic (PPG) sensor and accelerometer sensor. In addition, the WCD comprises a controller configured to monitor an output of the at least one non-invasive physiologic sensor for detecting a change in a health parameter of the subject. The change in health parameter of the subject can be indicative of one or more of a change in subject condition that may be a precursor to potential cardiac arrhythmia or a simultaneously occurring cardiac arrhythmia. In one embodiment, the photoplethysmographic (PPG) sensor is configured to monitor the change in health parameter as a function of arterial oxygen saturation, and the accelerometer sensor is configured to monitor the change in health parameter as a function of respiration and a lack of breathing.

Responsive to detecting the change, the controller activates an alarm for requesting a response from the subject within a predetermined period of time. Responsive to receiving the response from the subject within the predetermined period of time, the controller inhibits the means for electrically engaging the set of electrodes to the subject's skin. Otherwise, responsive to not receiving the response from the subject within the predetermined period of time, the controller initiates the means for electrically engaging the set of electrodes to the subject's skin.

In another embodiment, the set of electrodes comprises a single set of electrodes that is further at least operable to deliver a therapeutic shock to the subject. In this embodiment, the controller is further operable to deliver the therapeutic shock in response to an analysis of an ECG signal obtained after the set of electrodes is electrically engaged. In addition, in one embodiment, the set of electrodes comprises dry therapeutic electrodes with self-deploying gel that automatically deploys the gel prior to shock delivery, the therapeutic electrodes being configured for both obtaining of the ECG signal for assessment and shock determination and delivery of the therapeutic shock.

In a further embodiment, the set of electrodes comprises a first set of electrodes operable to sense the ECG signal from the subject and a second set of electrodes operable to deliver a therapeutic shock to the subject. In this embodiment, the engaging means electrically engages the first set of electrodes and the second set of electrodes to the subject's skin. In addition, the controller is operable to initiate the means for electrically engaging (i) the first set of electrodes and (ii) the second set of electrodes. The controller is further operable to obtain an ECG signal after the first set of electrodes is electrically engaged and deliver the therapeutic shock, in response to an analysis of the ECG signal obtained after the first set of electrodes is electrically engaged, after the second set of electrodes is electrically engaged. In one embodiment, the first set of electrodes comprises dry non-adhesive sensing electrodes and the second set of electrodes comprise dry therapeutic electrodes with self-deploying gel that automatically deploys the gel prior to shock delivery.

In accordance with another embodiment, the means for electrically engaging the set of electrodes includes a mechanism for disposing at least one conductive portion of each electrode of the set of electrodes between a non-conductive contact position and a conductive contact position. Responsive to being in the non-conductive contact position, the at least one electrically conductive portion of each electrode of the set of electrodes does not physically engage the set of electrodes for electrical contact to the subject's skin. In addition, responsive to being in the conductive contact position, the at least one electrically conductive portion of each electrode of the set of electrodes physically engages the set of electrodes for electrical contact to the subject's skin.

In yet another embodiment, the WCD further comprises an alarm module coupled to the controller for providing the alarm as activated by the controller. The alarm includes at least one or more of an audible, tactile, or visible alarm. In addition, the WCD comprises a user interface coupled to the controller for receiving the response from the subject. Furthermore, the WCD comprises a wearable garment; wherein the set of electrodes is disposed on at least one surface of the wearable garment adjacent the subject's skin in response to being worn by the subject. In a still further embodiment, the WCD comprises a means for communicating to a remote device, via at least one or more of wireless and wired communication, an occurrence of a therapeutic shock delivery with the set of electrodes.

According to another embodiment, a method of implementing a wearable cardioverter defibrillator (WCD) comprises configuring a set of electrodes for placement on a subject. The set of electrodes are at least operable to sense an ECG signal from the subject. In addition, the method comprises configuring at least one non-invasive physiologic sensor for placement on the subject, wherein the at least one non-invasive physiologic sensor comprises one or more of a photoplethysmographic (PPG) sensor and accelerometer sensor. The method further comprises monitoring, via a controller, an output of the at least one non-invasive physiologic sensor for detecting a change in a health parameter of the subject being indicative of one or more of a change in subject condition that may be a precursor to potential cardiac arrhythmia or a simultaneously occurring cardiac arrhythmia. The photoplethysmographic (PPG) sensor is configured to monitor the change in health parameter as a function of arterial oxygen saturation. The accelerometer sensor is configured to monitor the change in health parameter as a function of respiration and a lack of breathing.

Responsive to detecting the change, the method includes activating, via the controller, an alarm for requesting a response from the subject within a predetermined period of time, wherein (i) responsive to receiving the response from the subject within the predetermined period of time, inhibiting, via the controller, an electrical engagement of the set of electrodes to the subject's skin, and (ii) responsive to not receiving the response from the subject within the predetermined period of time, initiating, via the controller, a control signal for electrically engaging the set of electrodes, via an electrical engagement mechanism, to the subject's skin.

In one embodiment, the step of configuring the set of electrodes further includes configuring a single set of electrodes for being at least operable to deliver a therapeutic shock to the subject, further comprising delivering the therapeutic shock in response to an analysis of an ECG signal obtained after the set of electrodes is electrically engaged.

In another embodiment of the method, the step of configuring the set of electrodes comprises configuring a first set of electrodes operable to sense the ECG signal from the subject and a second set of electrodes operable to deliver a therapeutic shock to the subject. In addition, the step of electrically engaging comprises electrically engaging the first set of electrodes and the second set of electrodes to the subject's skin. Furthermore, initiating the control signal further comprises for electrically engaging (i) the first set of electrodes and (ii) the second set of electrodes, for obtaining an ECG signal after the first set of electrodes is electrically engaged and for delivering the therapeutic shock, in response to an analysis of the ECG signal obtained after the first set of electrodes is electrically engaged, after the second set of electrodes is electrically engaged. In one embodiment, the first set of electrodes comprises dry non-adhesive sensing electrodes and the second set of electrodes comprises dry therapeutic electrodes with self-deploying gel that automatically deploys the gel prior to shock delivery.

According to yet another embodiment, the method comprises electrically engaging the set of electrodes which includes disposing at least one conductive portion of each electrode of the set of electrodes between a non-conductive contact position and a conductive contact position. Responsive to being in the non-conductive contact position, the at least one electrically conductive portion of each electrode of the set of electrodes does not physically engage the set of electrodes for electrical contact to the subject's skin. In addition, Responsive to being in the conductive contact position, the at least one electrically conductive portion of each electrode of the set of electrodes physically engages the set of electrodes for electrical contact to the subject's skin. In a further embodiment, the set of electrodes comprises dry therapeutic electrodes with self-deploying gel that automatically deploys the gel prior to shock delivery, the therapeutic electrodes being configured for both obtaining of the ECG signal for assessment and shock determination and delivery of the therapeutic shock.

In a yet another embodiment, the method further comprises using an alarm module for providing the alarm as activated, wherein the alarm includes at least one or more of an audible, tactile, or visible alarm. A user interface is provided for receiving the response from the subject. In addition, the method further includes disposing the set of electrodes on at least one surface of a wearable garment adjacent the subject's skin in response to being worn by the subject. Still further, the method includes communicating to a remote device, via at least one or more of wireless and wired communication, an occurrence of a therapeutic shock delivery with the set of electrodes.

Still further advantages and benefits will become apparent to those of ordinary skill in the art upon reading and understanding the following detailed description.

The embodiments of the present disclosure may take form in various components and arrangements of components, and in various steps and arrangements of steps. Accordingly, the drawings are for purposes of illustrating the various embodiments and are not to be construed as limiting the embodiments. In the drawing figures, like reference numerals refer to like elements. In addition, it is to be noted that the figures may not be drawn to scale.

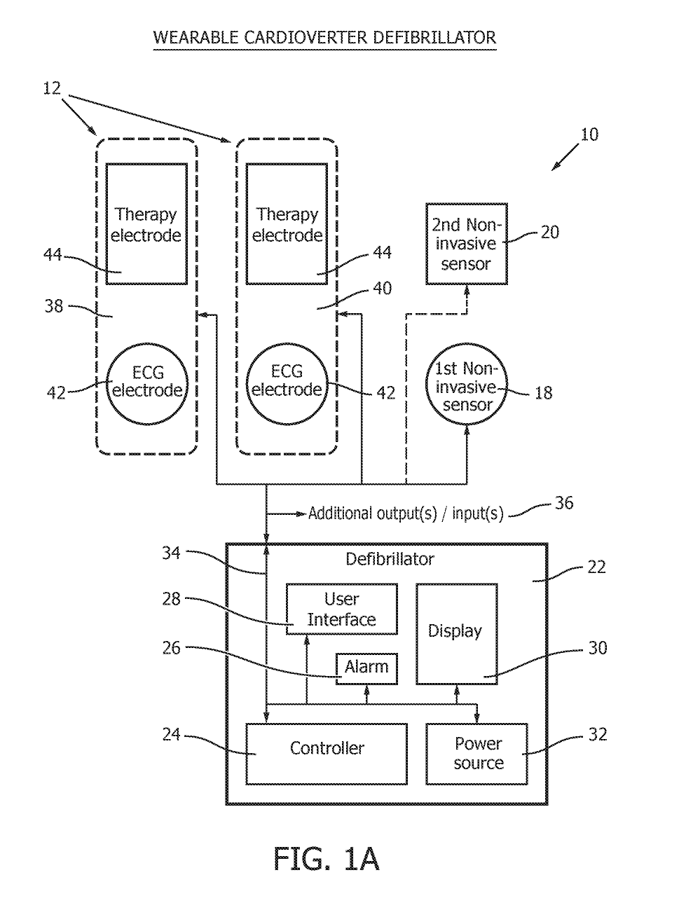

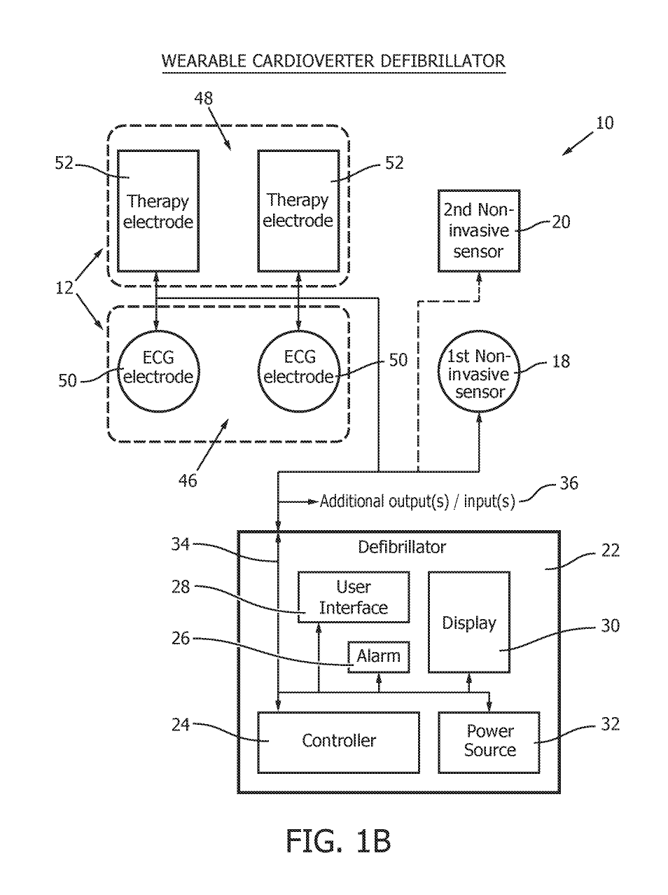

FIGS. 1A and 1B comprise block diagram views of a wearable cardioverter defibrillator (WCD) according to various embodiments of the present disclosure;

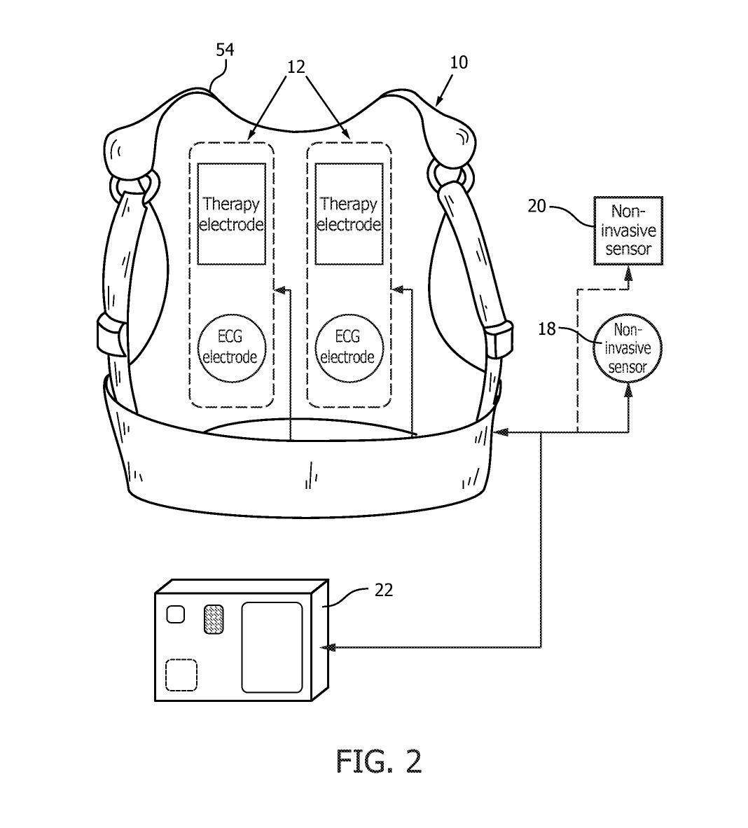

FIG. 2 is a perspective image and partial block diagram view of a WCD according to an embodiment of the present disclosure;

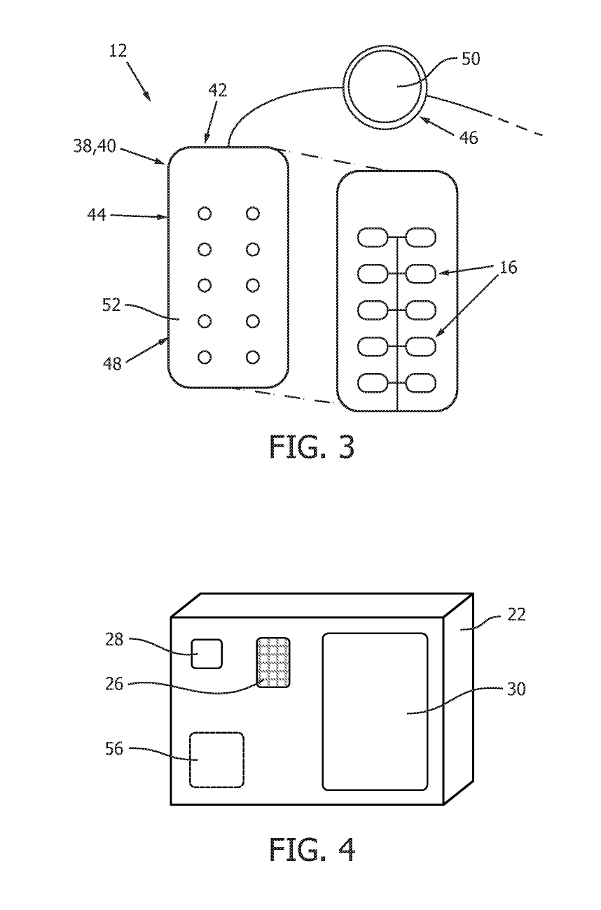

FIG. 3 is a perspective image view of several components of the set of electrodes in a WCD according to various embodiments of the present disclosure;

FIG. 4 is a perspective image view of an electronic control module for the WCD according to an embodiment of the present disclosure;

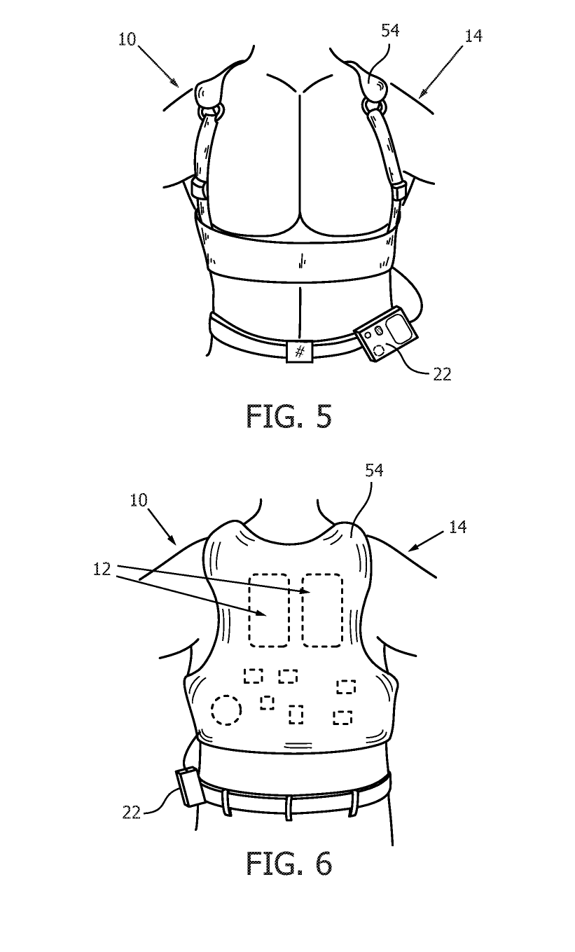

FIG. 5 is a front perspective image view of a WCD with the electronic control module being worn by a subject according to an embodiment of the present disclosure;

FIG. 6 is a rear perspective image view of a WCD with the electronic control module being worn by a subject according to an embodiment of the present disclosure; and

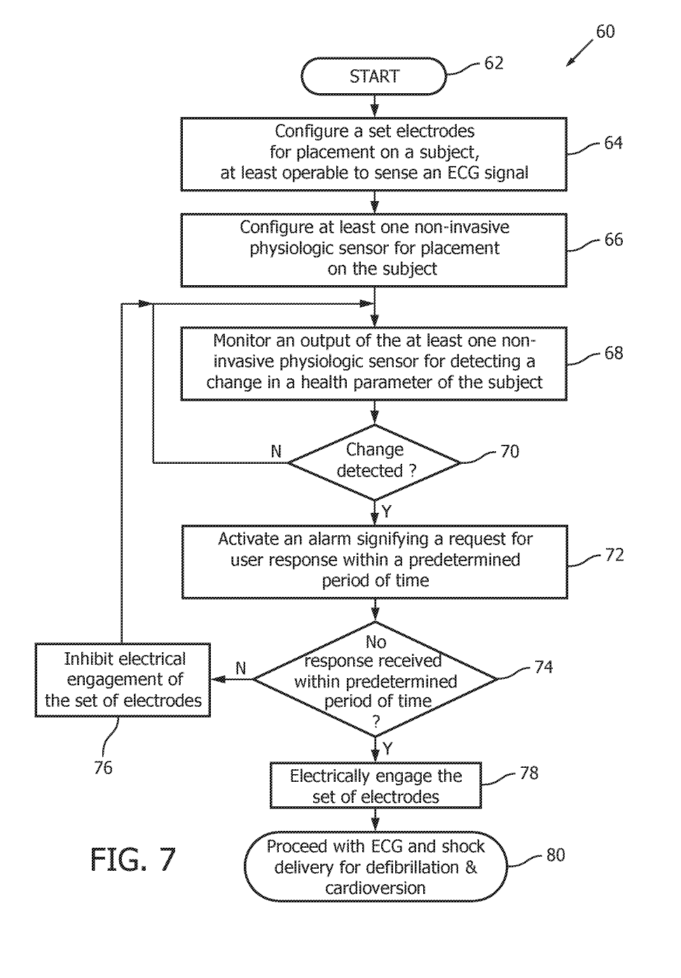

FIG. 7 is a flow diagram view illustrating a method of implementing a wearable cardioverter defibrillator (WCD) according to an embodiment of the present disclosure.

The embodiments of the present disclosure and the various features and advantageous details thereof are explained more fully with reference to the non-limiting examples that are described and/or illustrated in the drawings and detailed in the following description. It should be noted that the features illustrated in the drawings are not necessarily drawn to scale, and features of one embodiment may be employed with other embodiments as the skilled artisan would recognize, even if not explicitly stated herein. Descriptions of well-known components and processing techniques may be omitted so as to not unnecessarily obscure the embodiments of the present disclosure. The examples used herein are intended merely to facilitate an understanding of ways in which the embodiments of the present may be practiced and to further enable those of skill in the art to practice the same. Accordingly, the examples herein should not be construed as limiting the scope of the embodiments of the present disclosure, which is defined solely by the appended claims and applicable law.

It is understood that the embodiments of the present disclosure are not limited to the particular methodology, protocols, devices, apparatus, materials, applications, etc., described herein, as these may vary. It is also to be understood that the terminology used herein is used for the purpose of describing particular embodiments only, and is not intended to be limiting in scope of the embodiments as claimed. It must be noted that as used herein and in the appended claims, the singular forms “a,” “an,” and “the” include plural reference unless the context clearly dictates otherwise.

Unless defined otherwise, all technical and scientific terms used herein have the same meanings as commonly understood by one of ordinary skill in the art to which the embodiments of the present disclosure belong. Preferred methods, devices, and materials are described, although any methods and materials similar or equivalent to those described herein can be used in the practice or testing of the embodiments.

The embodiments of the present disclosure relate to non-electrode sensing of a patient parameter that indicates a potential cardiac problem. The sensing initiates the deployment of electrodes for more detailed diagnosis. The benefit of these embodiments is that a patient does not have to suffer with messy or uncomfortable electrodes on their skin on a continuous basis, which has been a significant compliance problem with existing wearable defibrillators.

As will be discussed further herein regarding the WCD of the present disclosure, an initial patient assessment is performed through alternative non-invasive physiologic sensors such as photoplethysmographic (PPG) or accerlerometery. Both PPG and accelerometers can be used to detect significant changes in a patient's state; PPG is well known to detect arterial oxygen saturation and accelerometers have been used to detect respiration. Both are easy to use, inexpensive and non-invasive. Importantly, both can be made to be comfortable to wear for long periods of time. In particular, PPG that uses green light and is less sensitive to noise. A sensor such as these in the WCD would monitor the general health of the patient. If a fatal rhythm were to occur, then the non-invasive sensor would identify this occurrence by a significant change in blood O₂ saturation (PPG) or lack of breathing (accelerometer). If a change in the patient's state was detected, an alarm would alert the patient giving the patient an opportunity to stop further action by the WCD. Lacking a patient response, the therapy electrodes would be applied via an electrically engaging mechanism (e.g., thru exploding gel) and a subsequent analysis of an ECG would take place thru the then electrically engaged electrodes to determine if a shockable rhythm was present. In one embodiment, the need for dry sensing electrodes can be eliminated.

Stated in a different manner, according to another embodiment of the present disclosure, the method includes initially monitoring a vital sign other than, or instead of, an ECG. As a result, the method advantageously provides for enabling a much more comfortable way to monitor a health parameter of a person, e.g., continuously monitor a plethsmography signal (or a respiration rate via an accelerometer, etc.) in a mostly healthy person. The vital sign monitoring doesn't have to be that difficult to do because the WCD will be looking for a healthy signal 99.9% of the time. If the pulse (or respiration or other non-ECG signal) indicates a problem, the WCD garment sounds an alarm to alert the wearer. If the wearer does not respond to the alarm, then gel pads (or other electrical engaging mechanisms) of the wearable garment are activated (i.e., electrically engaged with the user's skin) and only then is the ECG analyzed, whereupon defibrillation proceeds if needed. In addition, in one embodiment, the set of electrodes can include two combined ECG monitoring and defibrillation electrodes, each electrode of the single set being used for both ECG monitoring and defibrillation (i.e., no second set of electrodes). Accordingly, this results in providing a much more comfortable garment.

Referring now to FIG. 1A, there is shown a block diagram view of a wearable cardioverter defibrillator (WCD) 10 according to one embodiment of the present disclosure. The wearable cardioverter defibrillator 10 includes a set of electrodes 12 configured for placement on a subject 14 (FIGS. 5 and 6). The set of electrodes 12 is at least operable to sense an ECG signal from the subject. As will be discussed herein with reference to FIG. 3, the WCD 10 includes means for electrically engaging 16 the set of electrodes to the subject's skin. The WCD 10 further includes at least one non-invasive physiologic sensor, 18 and/or 20, configured for placement on the subject, wherein the at least one non-invasive physiologic sensor comprises one or more of a photoplethysmographic (PPG) sensor 18 and accelerometer sensor 20.

The WCD 10 further includes a cardioverter defibrillator control module 22 that comprises a controller 24, an alarm 26, a user interface 28, a display 30, and a power source 32. The control module 22 further provides and receives various signals between components of the WCD via signal/power lines, which are generally represented via reference numeral 34. The control module 22 further includes additional output(s) and/or input(s) 36 as may be required for a given wearable cardioverter defibrillator implementation.

The WCD 10 further includes a controller 24 configured to monitor an output of the at least one non-invasive physiologic sensor, 18 and/or 20, for detecting a change in a health parameter of the subject. Controller 24 comprises any suitable processor, microcontroller, or computer for executing the various functions of the embodiments disclosed herein. In particular, one or more output of non-invasive sensors 18 and/or 20 is monitored for detecting a change in health parameter of the subject that is indicative of one or more of a change in subject condition that may be (i) a precursor to potential cardiac arrhythmia or (ii) a simultaneously occurring cardiac arrhythmia. Responsive to detecting the change, the controller 24 activates an alarm 26 for requesting a response from the subject within a predetermined period of time. Subsequent to activating the alarm 26 and responsive to receiving the response from the subject within the predetermined period of time, the controller 24 inhibits an activation of the means for electrically engaging 16 the set of electrodes 12 to the subject's skin. In addition, subsequent to activating the alarm 26 and responsive to not receiving the response from the subject within the predetermined period of time, the controller 24 initiates the means for electrically engaging 16 the set of electrodes 12 to the subject's skin.

In the embodiment illustrated in FIG. 1A, the set of electrodes 12 comprises a single set of two electrodes 38 and 40. Each electrode (38,40) includes a combined electrode for being at least operable to sense an ECG signal 42 from the subject and further being at least operable to deliver a therapeutic shock 44 to the subject. In addition, the controller 24 is further operable to deliver the therapeutic shock in response to an analysis of an ECG signal obtained after the set of electrodes is electrically engaged.

With reference now to FIG. 1B, there is shown a block diagram view of a wearable cardioverter defibrillator (WCD) 10 according to another embodiment of the present disclosure. The embodiment of FIG. 1B is similar to that of FIG. 1A with the following differences. The set of electrodes 12 comprises a first set of electrodes 46 operable to sense the ECG signal from the subject and a second set of electrodes 48 operable to deliver a therapeutic shock to the subject. The first set of electrodes 46 comprises ECG electrodes 50 and the second set of electrodes 48 comprises therapeutic shock electrodes 52. As will be understood further herein, with at least reference to FIG. 3, the engaging means 16 electrically engages the first set of electrodes 46 and the second set of electrodes 48 to the subject's skin. In operation, the controller 24 is operable to initiate the means for electrically engaging 16 (i) the first set of electrodes 46 and (ii) the second set of electrodes 48. The controller 24 is further operable to obtain an ECG signal after the first set of electrodes 46 is electrically engaged and deliver the therapeutic shock, in response to an analysis of the ECG signal obtained after the first set of electrodes 46 is electrically engaged, after the second set of electrodes 48 is electrically engaged.

Turning now to FIG. 2, there is shown a perspective image and partial block diagram view of a WCD 10 according to an embodiment of the present disclosure. The embodiment of FIG. 2 is similar to that of FIGS. 1A and 1B with the following differences. WCD 10 includes a wearable garment 54 which can comprise any suitable durable fabric and/or material, and of an appropriate size, for being worn comfortably by a subject for extended periods of time (e.g., ˜24 hours per day, 7 days per week). The wearable garment 54 should also comprise a washable garment. Accordingly, the wearable garment 54, and thus the WCD 10, is intended to be worn mostly all the time, except when bathing. The set of electrodes 12 is incorporated within the wearable garment 54, and in particular, on an inside portion of the garment. In other words, the set of electrodes is disposed on at least one surface of the wearable garment adjacent the subject's skin in response to being worn by the subject. The set of electrodes 12 are further removable, as needed, e.g., for replacement and/or for washing of the wearable garment 54. When worn by a subject, the set of electrodes 12 is disposed for being adjacent the subject's skin in preparation for use as disclosed herein, further according to the requirements of a given cardioverter defibrillation implementation.

With reference now to FIG. 3, a perspective image view of several components of the set of electrodes 12 in the WCD 10 according to various embodiments of the present disclosure is shown. In one embodiment, the set of electrodes 12 comprises a single set of two electrodes 38,40 (only one electrode 38,40 of the set is shown in the figure). Each electrode (38,40) includes a combined electrode for being at least operable to sense an ECG signal 42 from the subject and further being at least operable to deliver a therapeutic shock 44 to the subject. In another embodiment, the set of electrodes 12 comprises a first set of electrodes 46 (only one electrode 50 of the first set is shown in the figure) operable to sense the ECG signal from the subject and a second set of electrodes 48 (only one electrode 52 of the second set is shown in the figure) operable to deliver a therapeutic shock to the subject.

With reference still to FIG. 3, the set of electrodes 12 include means for electrically engaging 16 the set of electrodes to the subject's skin. In one embodiment, the means for electrically engaging 16 the set of electrodes 12 comprises a self-deploying gel that automatically deploys the gel in response to an activation signal provided by the controller 24, as discussed herein above. As shown in FIG. 3, in one embodiment, the self-deploying gel is configured within a plurality of caps or caplets disposed and arranged for overlying corresponding electrical contact points of a respective electrode. The electrically engaging means 16 thus includes a mechanism for disposing at least one conductive portion of each electrode of the set of electrodes 12 between a non-conductive contact position and a conductive contact position, wherein responsive to being in the non-conductive contact position, the at least one electrically conductive portion of each electrode of the set of electrodes does not physically engage the set of electrodes for electrical contact to the subject's skin, and wherein responsive to being in the conductive contact position, the at least one electrically conductive portion of each electrode of the set of electrodes physically engages the set of electrodes for electrical contact to the subject's skin. Other implementations of a mechanism for electrically engaging the set of electrodes to the subject's skin are contemplated, for example, including a fluid or pressurized bladder arrangement for deploying the electrodes for electrical contact to the subject's skin.

In one embodiment, the set of electrodes 12 comprises dry therapeutic electrodes with self-deploying gel that automatically deploys the gel prior to shock delivery, the therapeutic electrodes being configured for both obtaining of the ECG signal for assessment and shock determination and delivery of the therapeutic shock. In another embodiment, the first set of electrodes 46 comprises dry non-adhesive sensing electrodes and the second set of electrodes 48 comprise dry therapeutic electrodes with self-deploying gel that automatically deploys the gel prior to shock delivery.

With reference now to FIG. 4, a perspective image view of an electronic control module 22, also referred to as a cardioverter defibrillator control module, for the WCD 10 according to an embodiment of the present disclosure is shown. The electronic control module 22 is configured for being worn by the subject, for example, being clipped to a belt, supported via a shoulder strap, or other suitable method. Also, with reference to FIGS. 1A and 1B, the WCD 10 includes an alarm module 26 coupled to the controller 24 for providing the alarm as activated by the controller. The alarm includes at least one or more of an audible, tactile, or visible alarm. In addition, the electronic control module 22 includes a user interface 28 coupled to the controller 24 for receiving the response from the subject. In one embodiment, the user interface 28 comprises a reset button. In another embodiment, the user interface 28 can comprise a touch screen display. Other forms of user interface are also contemplated, such as a voice command interface. Still further, the electronic control module 22 includes a means for communicating 56 to a remote device (not shown), via at least one or more of wireless and wired communication, an occurrence of a therapeutic shock delivery with the set of electrodes. The communicating means 56 (FIG. 4) can comprise any suitable transmitter/receiver (Tx/Rx) coupled to controller 24 (FIGS. 1A and 1B), for implementing a desired communication to/from a remote device (not shown). Such a desired communication may include an emergency type interface or similar for communicating with emergency medical professionals.

Turning now to FIG. 5, a front perspective image view of a WCD 10 with the electronic control module 22 being worn by a subject 14 according to an embodiment of the present disclosure is shown. In this illustration, the electronic control module 22 is worn by attaching the module to the subject's belt. FIG. 6 shows a rear perspective image view of the WCD 10 with the electronic control module 22 being worn by a subject 14. In addition, the set of electrodes 12 is illustrated via dashed lines, indicative of being on an inside surface of the wearable garment 54 and adjacent to the subject's skin. Other components (e.g., electrodes, wires, sensors, etc.) are also illustrated in dashed lines.

With reference now to FIG. 7, a flow diagram view illustrating a method 60 of implementing a wearable cardioverter defibrillator (WCD) according to an embodiment of the present disclosure shall be described. Upon initialization at start (Step 62), the method begins (Step 64) with configuring a set of electrodes for placement on a subject, wherein the set of electrodes is at least operable to sense an ECG signal. The method continues (Step 66) with configuring at least one non-invasive physiologic sensor for placement on the subject, via wearing or other suitable comfortable manner. In a next step, the method includes monitoring an output of the at least one non-invasive physiologic sensor for detecting a change in a health parameter of the subject (Step 68). The method proceeds with a query of whether or not a change been detected (Step 70). If no change has been detected, then the method loops back to monitoring the output of the at least one non-invasive physiologic sensor (Step 68). However, if a change has been detected, the method proceeds by activating an alarm signifying a request for a user response within a predetermined period of time (Step 72). The method proceeds with a query of whether NO user response has been received within the predetermined period of time (Step 74). Responsive to a user response being received within the period of time, the method proceeds by inhibiting an electrical engagement of the set of electrodes (Step 76) and subsequently looping back to monitoring the output of the at least one non-invasive physiologic sensor (Step 68). However, if NO response a change has been detected, the method proceeds with electrically engaging the set of electrodes (Step 78). Subsequent to electrically engaging the set of electrodes, the method proceeds (Step 80) with ECG and shock delivery for defibrillation and cardioversion, as necessary, for the given situation.

Although only a few exemplary embodiments have been described in detail above, those skilled in the art will readily appreciate that many modifications are possible in the exemplary embodiments without materially departing from the novel teachings and advantages of the embodiments of the present disclosure. For example, actual placement of the electrodes and sensors (PPG and accelerometers as well as therapeutic and sensing electrodes) is not limited to that which is shown in the figures and described in the text herein. The embodiments of the present disclosure also cover implementations where the sensors are actually placed in optimal locations for a given WCD application. For example, the PPG/accelerometer sensor may be on the wrist of a patient or worn around the neck (like a pendant) or embedded in the garment or placed in another location. Also, the active (i.e., shocking/therapeutic electrodes) need to be in a suitable configuration that covers the heart, e.g., one on the back (posterior) and one on the front (anterior). In one embodiment, one active electrode could be located on a belt portion of a garment that comes around the front of the patient, so that the therapy to the heart is applied between the active electrodes. Other electrode configurations may be possible for use in the WCD. Accordingly, all such modifications are intended to be included within the scope of the embodiments of the present disclosure as defined in the following claims. In the claims, means-plus-function clauses are intended to cover the structures described herein as performing the recited function and not only structural equivalents, but also equivalent structures.

In addition, any reference signs placed in parentheses in one or more claims shall not be construed as limiting the claims. The word “comprising” and “comprises,” and the like, does not exclude the presence of elements or steps other than those listed in any claim or the specification as a whole. The singular reference of an element does not exclude the plural references of such elements and vice-versa. One or more of the embodiments may be implemented by means of hardware comprising several distinct elements, and/or by means of a suitably programmed computer. In a device claim enumerating several means, several of these means may be embodied by one and the same item of hardware. The mere fact that certain measures are recited in mutually different dependent claims does not indicate that a combination of these measures cannot be used to an advantage.

1. A wearable cardioverter defibrillator (WCD) comprising:
a set of electrodes configured for placement on a subject, the set of electrodes at least operable to sense an ECG signal from the subject;
means for electrically engaging the set of electrodes to the subject's skin;
at least one non-invasive physiologic sensor configured for placement on the subject, wherein the at least one non-invasive physiologic sensor comprises one or more of a photoplethysmographic (PPG) sensor and accelerometer sensor; and
a controller configured to monitor an output of said at least one non-invasive physiologic sensor for detecting a change in a health parameter of the subject being indicative of one or more of a change in subject condition that may be a precursor to potential cardiac arrhythmia or a simultaneously occurring cardiac arrhythmia, wherein responsive to detecting the change, said controller activates an alarm for requesting a response from the subject within a predetermined period of time, wherein (i) responsive to receiving the response from the subject within the predetermined period of time, said controller inhibits the means for electrically engaging the set of electrodes to the subject's skin, and (ii) responsive to not receiving the response from the subject within the predetermined period of time, said controller initiates the means for electrically engaging the set of electrodes to the subject's skin,
wherein the set of electrodes comprises a first set of electrodes operable to sense the ECG signal from the subject and a second set of electrodes operable to deliver a therapeutic shock to the subject,
wherein the engaging means electrically engages the first set of electrodes and the second set of electrodes to the subject's skin, and
wherein the controller is operable to initiate the means for electrically engaging (i) the first set of electrodes and (ii) the second set of electrodes, the controller further operable to obtain an ECG signal after the first set of electrodes is electrically engaged and deliver the therapeutic shock, in response to an analysis of the ECG signal obtained after the first set of electrodes is electrically engaged, after the second set of electrodes is electrically engaged.

2. The WCD of claim 1, wherein the set of electrodes comprises a single set of electrodes that is further at least operable to deliver a therapeutic shock to the subject, and wherein the controller is further operable to deliver the therapeutic shock in response to an analysis of an ECG signal obtained after the set of electrodes is electrically engaged.

3. (canceled)

4. The WCD of claim 1, wherein the means for electrically engaging the set of electrodes includes a mechanism for disposing at least one conductive portion of each electrode of the set of electrodes between a non-conductive contact position and a conductive contact position, wherein responsive to being in the non-conductive contact position, the at least one electrically conductive portion of each electrode of the set of electrodes does not physically engage the set of electrodes for electrical contact to the subject's skin, and wherein responsive to being in the conductive contact position, the at least one electrically conductive portion of each electrode of the set of electrodes physically engages the set of electrodes for electrical contact to the subject's skin.

5. The WCD of claim 2, further wherein the set of electrodes comprises dry therapeutic electrodes with self-deploying gel that automatically deploys the gel prior to shock delivery, the therapeutic electrodes being configured for both obtaining of the ECG signal for assessment and shock determination and delivery of the therapeutic shock. operable to sense an ECG signal from the subject (Step 64);
configuring at least one non-invasive physiologic sensor for placement on the subject, wherein the at least one non-invasive physiologic sensor comprises one or more of a photoplethysmographic (PPG) sensor and accelerometer sensor (Step 66); and
monitoring, via a controller, an output of said at least one non-invasive physiologic sensor for detecting a change in a health parameter (Step 68) of the subject being indicative of one or more of a change in subject condition that may be a precursor to potential cardiac arrhythmia or a simultaneously occurring cardiac arrhythmia, wherein responsive to detecting the change, activating, via the controller, an alarm (Step 72) for requesting a response from the subject within a predetermined period of time, wherein (i) responsive to receiving the response from the subject within the predetermined period of time, inhibiting (Step 76), via the controller, an electrical engagement of the set of electrodes to the subject's skin, and (ii) responsive to not receiving the response from the subject within the predetermined period of time, initiating (Step 78), via the controller, a control signal for electrically engaging the set of electrodes, via an electrical engagement mechanism, to the subject's skin,
wherein configuring the set of electrodes (Step 64) comprises configuring a first set of electrodes operable to sense the ECG signal from the subject and a second set of electrodes operable to deliver a therapeutic shock to the subject,
wherein electrically engaging (Step 78) comprises electrically engaging the first set of electrodes and the second set of electrodes to the subject's skin, and
wherein initiating the control signal further comprises for electrically engaging (i) the first set of electrodes and (ii) the second set of electrodes, for obtaining an ECG signal after the first set of electrodes is electrically engaged, in response to an analysis of the ECG signal obtained after the first set of electrodes is electrically engaged, after the second set of electrodes is electrically engaged.

12. The method of claim 11, wherein configuring the set of electrodes (Step 64) further includes configuring a single set of electrodes for being at least operable to deliver a therapeutic shock to the subject, further comprising delivering the therapeutic shock in response to an analysis of an ECG signal obtained after the set of electrodes is electrically engaged.

13. (canceled)

14. The method of claim 11, wherein electrically engaging (Step 78) the set of electrodes includes disposing at least one conductive portion of each electrode of the set of electrodes between a non-conductive contact position and a conductive contact position, wherein responsive to being in the non-conductive contact position, the at least one electrically conductive portion of each electrode of the set of electrodes does not physically engage the set of electrodes for electrical contact to the subject's skin, and wherein responsive to being in the conductive contact position, the at least one electrically conductive portion of each electrode of the set of electrodes physically engages the set of electrodes for electrical contact to the subject's skin.

15. The method of claim 12, further wherein the set of electrodes comprises dry therapeutic electrodes with self-deploying gel that automatically deploys the gel prior to shock delivery, the therapeutic electrodes being configured for both obtaining of the ECG signal for assessment and shock determination and delivery of the therapeutic shock.

16. The method of claim 11, further wherein the first set of electrodes comprises dry non-adhesive sensing electrodes and the second set of electrodes comprises dry therapeutic electrodes with self-deploying gel that automatically deploys the gel prior to shock delivery.

17. The method of claim 11, further comprising:
providing an alarm module for providing the alarm as activated, wherein the alarm includes at least one or more of an audible, tactile, or visible alarm; and
providing a user interface for receiving the response from the subject.

18. The method of claim 11, further comprising:
disposing the set of electrodes on at least one surface of a wearable garment adjacent the subject's skin in response to being worn by the subject.

19. The method of claim 11, further comprising:
communicating to a remote device, via at least one or more of wireless and wired communication, an occurrence of a therapeutic shock delivery with the set of electrodes.

20. The method of claim 11, wherein the photoplethysmographic (PPG) sensor is configured to monitor the change in health parameter as a function of arterial oxygen saturation, and wherein the accelerometer sensor is configured to monitor the change in health parameter as a function of respiration and a lack of breathing.
